(19) United States
(12) Patent Application Publication
(10) Pub. No.: US 2026/0324949 A1
(43) Pub. Date: Oct. 22, 2026

(54) INTRAVASCULAR ENDOSCOPY

(71) Applicant: Northwestern University, Evanston, IL (US)

(72) Inventors: Ahsun RIAZ, Evanston, IL (US); Riad SALEM, Evanston, IL (US); Abhinav TALWAR, Evanston, IL (US)

(21) Appl. No.: 19/113,873

(22) PCT Filed: Sep. 26, 2023
(86) PCT No.: PCT/US2023/075141

§ 371 Date: Mar. 20, 2025

Related U.S. Application Data
(60) Provisional application No. 63/377,125, filed on Sep. 26, 2022.

(51) Int. Cl. A61B 1/00; A61B 1/018; A61B 1/05; A61B 1/06; A61B 1/313

(52) U.S. Cl. CPC A61B 1/00082; A61B 1/018; A61B 1/051; A61B 1/0676; A61B 1/3137

(57) ABSTRACT

Provided herein are endoscopes with a transparent balloon mounted thereon that find use in angioscopy and cardioscopy. In particular, an endoscope-mounted balloon with a transparent lining that can be inflated with transparent liquid and apposed to the wall of a blood vessel or the heart allows visualization of the adjacent structures through the balloon.

### CROSS-REFERENCE TO RELATED APPLICATION

The present invention claims the benefit of U.S. Provisional Patent Application Ser. No. 63/377,125, filed Sep. 26, 2023, which is hereby incorporated by reference in its entirety

### FIELD

Provided herein are endoscopes with a transparent balloon mounted thereon that find use in angioscopy and cardioscopy. In particular, an endoscope-mounted balloon that can be inflated with transparent liquid and apposed to the wall of a blood vessel or the heart allows visualization of the adjacent structures through the balloon.

### BACKGROUND

Angioscopy and cardioscopy are performed as a part of highly invasive surgical procedures that require cardiac bypass to allow for segments of the heart and/or blood vessels (arteries and veins) to be void of blood to allow visualization of the walls. However, given the level of invasiveness of such procedures, cameras are rarely used within blood vessels and the heart for more routine and less invasive procedures.

### SUMMARY

Provided herein are endoscopes with a transparent balloon mounted thereon that find use in angioscopy and cardioscopy. In particular, an endoscope-mounted balloon that can be inflated with transparent liquid and apposed to the wall of a blood vessel or the heart allows visualization of the adjacent structures through the balloon.

In some embodiments, provided herein are endoscopes comprising a main body with a primary axis and a distal end; wherein the distal end is configured for insertion into a subject and placement adjacent to a treatment or diagnostic site within the subject; wherein the distal end comprises a camera and a light source; wherein the distal end comprises a transparent balloon; wherein when the balloon is filled with a transparent fluid, the balloon occupies a space between the camera the treatment or diagnostic site and an operator of the endoscope is capable of visualizing the treatment or diagnostic site through the balloon with the camera. In some embodiments, the distal end of the endoscope comprises the terminal 1-4 cm (e.g., 1 cm, 1.5 cm, 2 cm, 2.5 cm, 3 cm, 3.5 cm, 4 cm, or ranges therebetween) of the endoscope.

In some embodiments, the camera is oriented orthogonally to the primary axis of the endoscope. In some embodiments, the balloon is oriented orthogonally to the primary axis of the endoscope. In some embodiments, the balloon is on the same side of the endoscope as the camera. In some embodiments, the balloon surrounds the periphery of the distal end of the endoscope. In some embodiments, the light source is oriented orthogonally to the primary axis of the endoscope. In some embodiments, the light is oriented in the same direction as the camera. In some embodiments, the distal end further comprises one or more ports oriented orthogonally to the primary axis. In some embodiments, an endoscope further comprises a working channel extending from a port and through the balloon. In some embodiments, wires, instruments, devices, therapeutics, and/or materials can be transported from the interior of the endoscope to the treatment or diagnostic site through the working channel. In some embodiments, biological samples can be transported from the treatment or diagnostic site to the interior of the main body of the endoscope through the working channel. In some embodiments, the working channel oriented orthogonally to the primary axis. In some embodiments, the working channel is conical or funnel shaped, having a distal end oriented away from the main body with a greater diameter than a proximal end oriented toward the main body. In some embodiments, the working channel comprises one or more non-compliant rings oriented along its length to prevent collapse of the working channel under pressure from the filled balloon.

In some embodiments, the camera is positioned at the distal tip of the endoscope and oriented to view beyond the distal end of the endoscope. In some embodiments, the balloon is positioned at the distal tip of the endoscope and oriented to be filled beyond the distal end of the endoscope. In some embodiments, the light source is positioned at the distal tip of the endoscope and oriented to illuminate the treatment site beyond the distal end of the endoscope. In some embodiments, the light is oriented in the same direction as the camera. In some embodiments, the distal end further comprises one or more ports positioned at the distal tip of the endoscope. In some embodiments, an endoscope further comprises a working channel extending from a port and through the balloon. In some embodiments, wires, instruments, devices, therapeutics, and/or materials can be transported from the interior of the endoscope to the treatment or diagnostic site through the working channel. In some embodiments, biological samples can be transported from the treatment or diagnostic site to the interior of the main body of the endoscope through the working channel. In some embodiments, the working channel is oriented substantially parallel (e.g., +/−15°) to the primary axis. In some embodiments, the working channel is conical or funnel shaped, having a distal end oriented away from the main body with a greater diameter than a proximal end oriented toward the main body. In some embodiments, the working channel comprises one or more non-compliant rings oriented along its length to prevent collapse of the working channel under pressure from the filled balloon.

In some embodiments, provided herein are methods of viewing a treatment or diagnostic site within a blood vessel or cardiac location of a subject, the method comprising: (a) placing the distal end of an endoscope described herein adjacent to the treatment or diagnostic site; (b) filling the balloon with clear fluid, wherein the balloon fills the space between the endoscope and the treatment or diagnostic site; (c) illuminating the treatment or diagnostic site with the light source; and (d) observing the treatment or diagnostic site through the filled balloon with the camera. In some embodiments, the blood vessel is a vein. In some embodiments, the blood vessel is an artery. In some embodiments, when the balloon is filled, the balloon fills the space around the periphery of the endoscope.

### BRIEF DESCRIPTION OF THE DRAWINGS

FIG. 1. Drawing depicting a delivery sheath (20) with a guidewire (1) advanced through a blood vessel.

FIG. 2. Drawing depicting an exemplary balloon (2) which has been inflated with transparent fluid to displace the blood in the segment of blood vessel being analyzed. The scope component (10) has been advanced over the guidewire (1). A camera (3), light source (4), and working channel opening (5) are depicted on this exemplary embodiment.

FIG. 3. Drawing depicting exemplary endoscope (10) and balloon (2) components from a side view: camera (3), light source (4), and funnel shaped reinforced working channel (8) through the compliant balloon.

FIG. 4. Drawing depicting exemplary endoscope (10) components (front view): camera (3), light source (4), and funnel shaped reinforced working channel (8) through the compliant balloon.

FIG. 5. Drawing depicting an endoscope (10) with in-line guidewire withdrawn (white arrow) and a wire (11) deployed through the working channel (8) of the scope (10) advanced into a side branch blood vessel.

FIG. 6. Drawing depicting an exemplary endoscope (10) with the balloon completely deflated (white arrow) and the wire (11) through the working channel opening (8) of the scope advanced into a side branch. The scope can now be pulled over the wire maintaining access into the side branch.

FIG. 7A-C. Drawings depicting an exemplary endoscope. (A) Front view of an exemplary intravascular endoscope: wire (1) going through the endoscope; clear balloon (2) filled with clear fluid to serve as the medium to see through; camera (3); light source (4); working channel opening (5); channel to inflate/deflate the balloon (6); and opening to inflate/deflate the balloon (7). (B) Profile view of an exemplary endoscope: wire (1) going through the endoscope; clear balloon (2) filled with clear fluid to serve as the medium to see through; camera (3); working channel opening (5); and working channel (8). (C) Top view of an exemplary endoscope: wire going through the endoscope (1); clear balloon (2) filled with clear fluid to serve as the medium to see through; camera (3); and channel in the balloon to maintain a degree of blood flow while the balloon is inflated (9).

FIG. 8A-B. Side-view (A) and top-view (B) images of an exemplary endoscope (10) being advanced into a syringe (30) containing an opaque fluid (31).

FIG. 9A-B. (A) Image of an exemplary endoscope inserted into the opaque fluid within a syringe. (B) Image taken from a camera of the exemplary endoscope of 9A; nothing is visible except for the glare caused by endoscope's light.

FIG. 10A-B. (A) Image of an exemplary endoscope inserted into the opaque fluid within a syringe, with the clear balloon (2) expanded. (B) Image taken from a camera of the exemplary endoscope of 10A; the markings on the inner lining of syringe are visible.

### DETAILED DESCRIPTION

Provided herein are endoscopes with a transparent balloon mounted thereon that find use in angioscopy and cardioscopy. In particular, an endoscope-mounted balloon that can be inflated with transparent liquid and apposed to the wall of a blood vessel or the heart allows visualization of the adjacent structures through the balloon.

Provided herein are devices comprising an endoscope-mounted balloon that allows for visualization of blood vessels and the interior of the heart by angioscopy and cardioscopy. A transparent and compliant balloon (e.g., silicone or polyurethane) is mounted on the distal end of an endoscope. In some embodiments, the balloon is mounted on the side of the distal end of an endoscope. When the balloon is filled with a transparent fluid (e.g., gas or liquid), the balloon is apposed to the wall of the blood vessel or heart within which it resides. The wall can then be visualized through the transparent fluid and lining of the balloon. In some embodiments, the devices herein further allow for passage of wires through a channel in the balloon.

In some embodiments, devices provided herein are endoscopes, or more particularly angioscopes or cardioscopes. As such, the devices herein may comprise any features, components, materials, etc. understood in the field of endoscopes (e.g., angioscopes, cardioscopes, etc.). In some embodiments, the endoscopes herein comprise a light source and a camera. In some embodiments, endoscopes herein comprise components and features similar to known commercially available endoscopes, such as the SPYGLASS DISCOVER (Boston Scientific; Marlborough; MA).

In some embodiments, an endoscope herein may have one or more channels (e.g., lumens) within its body. In some embodiments, the channels allow materials (e.g., clear fluid to fill balloon) to be passed through the endoscope (e.g., from the control end to the distal end) and provide connections between the controls and the working components at the distal end of the endoscope (e.g., camera, light source, orthogonal working channel, wires, etc.).

In some embodiments, an endoscope comprises a guidewire channel. The guidewire channel runs the length of the endoscope along the primary axis, from the control end to the distal end, and extends out of the distal tip of the endoscope. A guidewire running through the guidewire channel allows the endoscope to be advanced along the guidewire to the treatment/diagnostic site.

In some embodiments, an endoscope comprises a secondary wire channel. The secondary wire channel runs the length of the endoscope along the primary axis, from the control end to the distal end, and extends out of the working side of the endoscope. In some embodiments, the secondary wire channel terminates at the working channel through the balloon.

The secondary wire channel provides a side channel to allow advancement of the wire/device through a working channel (e.g., through the balloon). In some embodiments, a working channel extends out of the distal tip of the endoscope (rather than a side). In such embodiments, a secondary wire may also extend out of the distal tip of the endoscope (rather than a side).

In some embodiments, an endoscope comprises a controller located at the proximal end (control end) of the endoscope. The endoscope extends from the controller to the distal end. In some embodiments, the controller remains outside of the subject with the distal end is inserted into the subject and at the treatment/diagnostic site. In some embodiments, the controller comprises components that allow the operator (clinician) to maneuver (e.g., steer, advance, etc.) the endoscope, fill/unfill the balloon, introduce materials to the treatment/diagnostic site, take samples, operate the camera, illuminate the treatment/diagnostic site, etc. In some embodiments, the controller is in electronic, mechanical, and/or fluid commination with the distal end of the endoscope (or components located at the distal end of the endoscope) via one or more channels running along the primary axis of the endoscope (e.g., through the endoscope).

The distal end of the endoscope is equipped with a balloon. In some embodiments, the balloon is located on the working side of the endoscope, which also comprises one or more of the camera, the light source, and a working channel (e.g., through the balloon). In some embodiments, the balloon is circumferential to the distal end of the endoscope. In some embodiments, the balloon envelopes the full circumference of the distal end of the endoscope. In some embodiments, the balloon envelopes at least 30% of the circumference (e.g., 30%, 40%, 50%, 60%, 70%, 80%, 90%, 100%) of the distal end of the endoscope, including the working side of the distal end of the endoscope. In some embodiments, the balloon extends from the distal tip of the endoscope (e.g., rather than a side of the endoscope); in such embodiments, one or more of the camera, the light source, a working channel (e.g., through the balloon), etc. may also extend from the distal tip of the endoscope (e.g., rather than a side of the endoscope).

In some embodiments, the balloon is transparent (e.g., at least 70% transparent (e.g., >70%, >75%, >80%, >90%, >95%)), for example, to the wavelengths of light emitted by the light source.

In some embodiments, the balloon is compliant, allowing it to expand to fill a blood vessel or cardiac space. In some embodiments, the balloon is sized to occlude a blood vessel or cardiac space, preventing blood from occupying (e.g., flowing) the space adjacent to the working side of the endoscope. In some embodiments, the filled balloon occupies the full blood vessel or cardiac space, thereby occluding blood flow through that space. In some embodiments, the balloon size and/or shape is selected based upon the diagnostic/treatment site. In some embodiments, the balloon has a length (e.g., in-line with the primary axis of the endoscope) of 0.25 cm to 3 cm (e.g., 0.25 cm, 0.5 cm, 0.75 cm, 1.0 cm, 1.25 cm, 1.5 cm, 1.75 cm, 2.0 cm, 2.25 cm, 2.5 cm, 2.75 cm, 3 cm, or sizes therebetween). In some embodiments, the balloon has a width or diameter (e.g., orthogonal to the primary axis of the endoscope) of 0.25 cm to 3 cm (e.g., 0.5 cm, 0.75 cm, 1.0 cm, 1.25 cm, 1.5 cm, 1.75 cm, 2.0 cm, 2.25 cm, 2.5 cm, 2.75 cm, 3 cm, or sizes therebetween). Larger or smaller balloons may find use in certain embodiments or diagnostic/treatment sites. In some embodiments, the balloon is capable of adopting the shape of the vessel/structure within which the treatment/diagnostic site resides. In some embodiments, the filled balloon apposes the wall of the vessel/structure.

In some embodiments, the balloon is filled with any suitable clear fluid. In some embodiments, the fluid is a gas, such as air, CO₂, N₂, etc. In some embodiments, the clear fluid is a liquid, such as water or saline.

In some embodiments, the balloon comprises a channel that runs through the balloon (e.g., substantially parallel (e.g., +/−15°) to the endoscope) and allows blood to flow through the channel when the balloon is filled.

In some embodiments, the balloon material can be any of polyesters, polyamides, nylon 12, nylon 11, polyamide 12, block copolymers of polyether and polyamide, PEBAX, polyurethanes, block copolymers of polyether and polyester, or any other materials suitable for construction of a compliant biocompatible balloons, such as those used for angioplasty or other balloon procedures. The materials selected for the balloon result in a transparent balloon, capable of allowing light to pass through the balloon (e.g., from the endoscope light source) and for the camera to view the treatment/diagnostic site through the balloon.

In some embodiments, light from the light source is capable of passing through the filled balloon and the fluid contents thereof. In some embodiments, the treatment/diagnostic site is visible through the filled balloon and the fluid contents thereof.

In some embodiments, fluid is passed through the length of the endoscope and to the balloon via balloon channel. In some embodiments, fluid source is located at the proximal end of the endoscope. In some embodiments, a pressure gauge measures and/or regulates the pressure within the balloon.

In some embodiments, an endoscope herein comprises one or more working channels. In some embodiments, a working channel permits one or more tools (e.g., ablation devices, cannulas, dissectors, electrodes, forceps, graspers, knot pushers, laser fibers, needle holders, suction and irrigation instruments, trocars, and other tools) to be passed through the endoscope to the distal end and to the exterior of the endoscope at the diagnostic/treatment site (e.g., out a side or the distal tip of the endoscope).

In particular, endoscopes herein comprise a working channel extending from a port on the working side (or tip) of the distal end of the endoscope through the balloon. This working channel (e.g., side working channel, distal-tip working channel, etc.) allows for advancement of wires/devices to the diagnostic/treatment site. In some embodiments, the working channel (e.g., side working channel, distal-tip working channel, etc.) is observable by the camera. In some embodiments, the distal end of the working channel (e.g., side working channel, distal-tip working channel, etc.) is observable by the camera. In some embodiments, the working channel is positioned (e.g., angled) to position a device or other element that has been advanced through the side working channel within view of the camera. In some embodiments, the working channel (e.g., side working channel, distal-tip working channel, etc.) is reinforced with one or more (e.g., 1, 2, 3, 4, 5, 6, 7, 8, 9, 10, or more) circular rings to maintain its shape (e.g., funneled shape) in a compliant balloon from the port on the scope to the edge of the balloon. In some embodiments, the working channel comprises a valve to prevent back-bleeding into the endoscope.

In some embodiments, a working channel extends from the distal end of the endoscope, orthogonal to the primary axis of the endoscope, through the balloon. In some embodiments, a working channel extends from the distal tip of the endoscope, substantially parallel (e.g., +/−15°) to the primary axis of the endoscope, through the balloon (e.g., beyond the tip of the endoscope). In some embodiments, the working channel (e.g., the distal end of the working channel, the space adjacent to the distal end of the working channel, etc. is capable of being illuminated by the light source of the endoscope and viewed through the camera of the endoscope.

In some embodiments, endoscopes herein comprise an image sensor within the distal tip that provides a field of view external to the endoscope. In some embodiments, the image sensor is a camera. In some embodiments, the image sensor is capable of acquiring still images or video. In some embodiments, the camera is positioned on the working side of the endoscope and directed substantially orthogonally (e.g., +/−15°) to the primary axis of the endoscope (e.g., beyond the tip of the endoscope). In some embodiments, the camera is positioned on the distal tip of the endoscope and directed substantially parallel (e.g., +/−15°) to the primary axis of the endoscope. In some embodiments, when the distal end of the endoscope is adjacent to the diagnostic/treatment site, and the balloon is filled and abutting the wall of the blood vessel or cardiac space, the diagnostic/treatment site and/or the distal end of the side working channel is within the field of view of the camera.

In some embodiments, endoscopes herein comprise a light source within the distal tip that is capable of illuminating the treatment/diagnostic site. In some embodiments, the light source is an LED or other type of light. In some embodiments, the light source is positioned on the working side of the endoscope and configured to direct light orthogonal to the primary axis of the endoscope. In some embodiments, the light source is positioned on the distal tip of the endoscope and configured to direct light parallel to the primary axis of the endoscope (e.g., beyond the tip of the endoscope). In some embodiments, the light source is positioned and oriented to illuminate the treatment/diagnostic site and/or the distal end of the side working channel.

In some embodiments, an endoscope herein is delivered through the blood vessels of a subject to the treatment/diagnostic site within a delivery sheath.

Endoscopes and components thereof may be constructed of any suitable materials, as would be understood by those in the field, such as metals (e.g., steel, stainless steel, tungsten, titanium, aluminum nickel, copper, gold, platinum, and alloys thereof), glass, insulators (e.g., fluorinated ethylene propylene (FEP), perfluoroalkoxy (PFA), polytetrafluoroethylene (PTFE), etc.), elastomeric or polymeric materials (e.g., silicone, polyamides, polyesters, polyetheretherketones, polyetherurethanes, polyimides, polytetrafluoroethylene, polyurethane epoxies, etc.), copolyester elastomers (e.g., ARNITEL), polyether block amide (e.g., PEBAX), polyether polyester block copolymers (e.g., HYTREL), polyolefin elastomers (e.g., ENGAGE), polyurethane elastomer (e.g., PELLETHANE), styrene block copolymers (e.g., EVOPRENE), styrene-butadiene block copolymers (e.g., STYROFLEX), styrene-ethylene-butylene-styrene block copolymers (e.g., KRATON), and thermoplastic vulcanizates (e.g., SANTOPRENE and GEOLAST), etc.), etc. Embodiments herein are not limited by the materials to construct the endoscopes and components herein.

In some embodiments, the distal end of an endoscope herein is inserted into an artery or vein of a subject and maneuvered to a diagnostic or treatment site within a blood vessel or a cardiac location of a subject. In some embodiments, the endoscope is inserted through and/or to the diagnostic or treatment site within an artery, such as an artery selected from: the aorta, the common carotid artery, the external carotid artery, the triangles of the neck, the internal carotid artery, the subclavian artery, the axillary artery, the brachial artery, the radial artery, the ulnar artery, the descending aorta, the thoracic aorta, the abdominal aorta, a common iliac artery (e.g., the hypogastric artery, the external iliac artery), the femoral artery, the popliteal artery, the anterior tibial artery, the arteria dorsalis pedis, the posterior tibial artery, etc. In some embodiments, the endoscope is inserted through and/or to the diagnostic or treatment site within a vein, such as a vein selected from: a jugular vein, a hepatic vein, a basilic vein, a saphenous vein, a cephalic vein, a superior vena cava, an inferior vena cava, a pulmonary vein, a subclavian vein, a common iliac vein, an axillary vein, a portal vein, a renal vein, a popliteal vein, a brachiocephalic vein, a posterior tibial vein, a vertebral vein, a median cubital vein, an anterior tibial vein, a retromandibular vein, a femoral vein, a vein from a pterygoid plexus, an azygos vein, a superior mesenteric vein, a posterior auricular vein, a superior sagittal sinus vein, a superior ophthalmic vein, an internal iliac vein, a superficial temporal vein, a coronary sinus, a diploic vein, an anterior jugular vein, a common facial vein, a nasofrontal vein, an occipital vein, an external iliac vein, a deep vein of the thigh, an inferior ophthalmic vein, a vein connecting to an inferior petrosal sinus, an inferior thyroid vein, a splenic vein, a median antebrachial vein, a vein connecting to a superior petrosal sinus, a middle thyroid vein, a deep facial vein, a vein connecting to an inferior sagittal sinus, a frontal vein, etc. In some embodiments, the endoscope is inserted through and/or to the diagnostic or treatment site within a cardiac location, such as: the right atrium, the left atrium, the right ventricle, the left ventricle, etc. Embodiments herein may not be limited by the diagnostic/treatment site, the location of insertion of the device into a subject, or the path of the endoscope from the insertion site to the diagnostic/treatment site.

Intravascular and/or intracardiac endoscopy finds use in, for example: diagnoses and providing wire access for intravascular dissection (e.g., differentiate between true and false lumens), diagnosis and endothelial biopsy of vasculitis, providing access (e.g., wire access) to vascular side branches (e.g., occluded side branches), coiling of aneurysms, evaluation of placed stents (e.g., stent grafts for aortic aneurysms), treatment (e.g., external compression) or observation of venous disease (e.g., thrombosis, etc.), treatment or observation of cardiac abnormalities, etc.

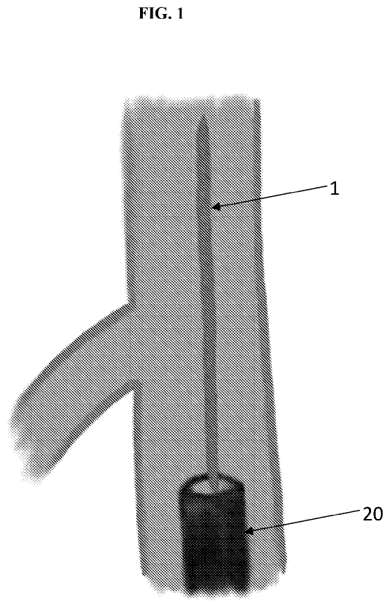

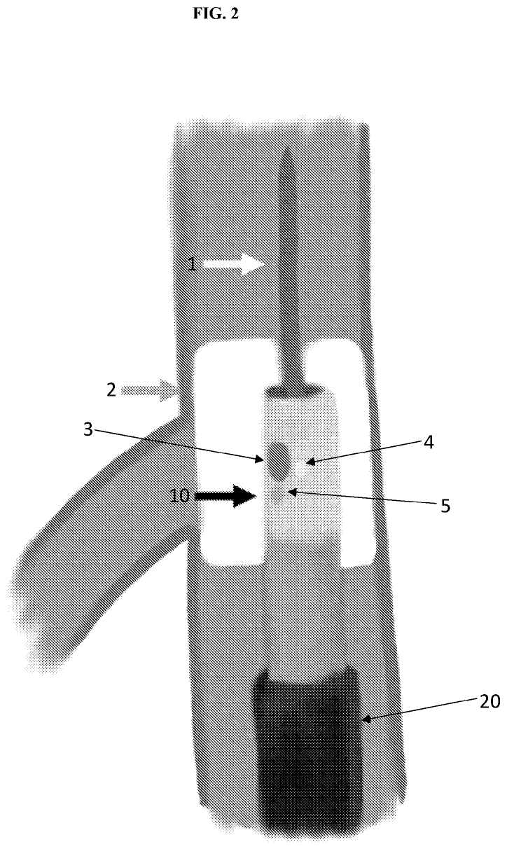

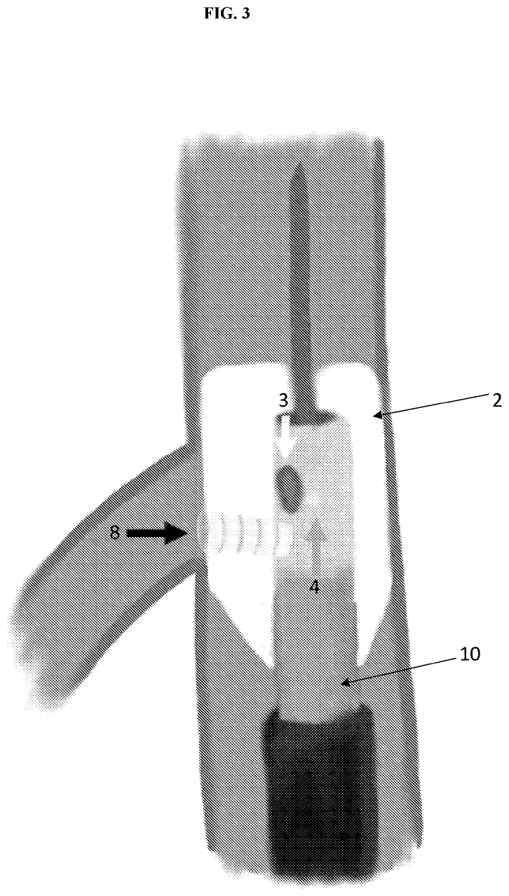

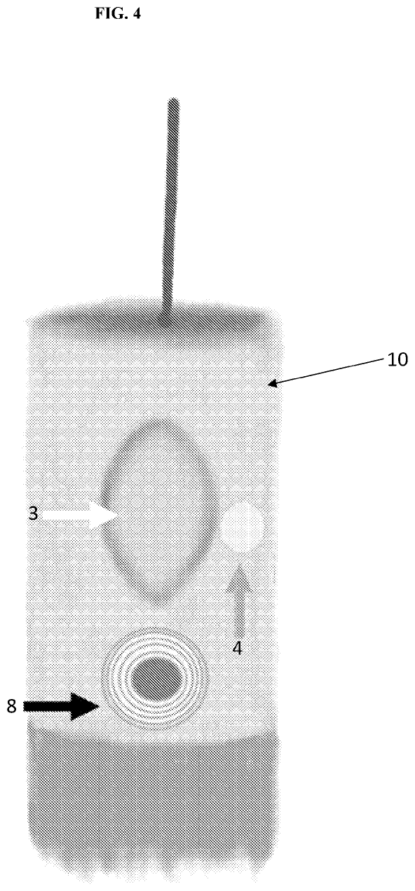

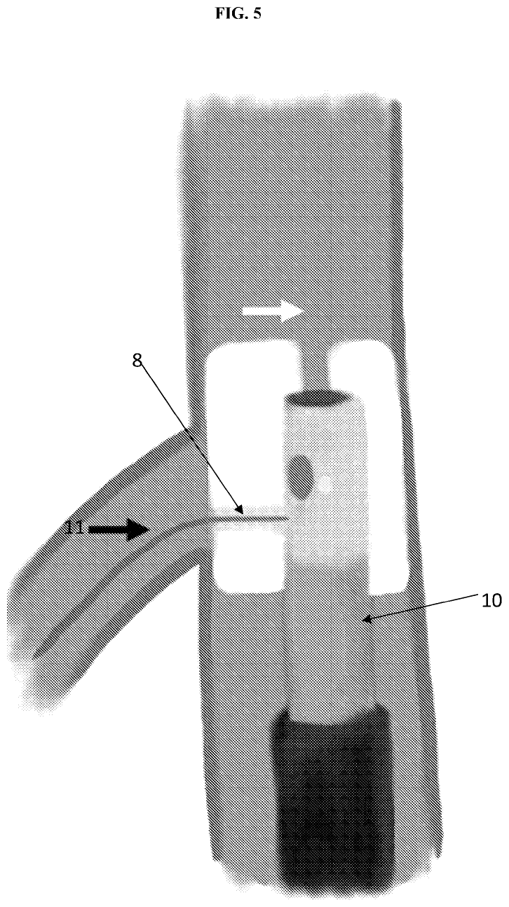

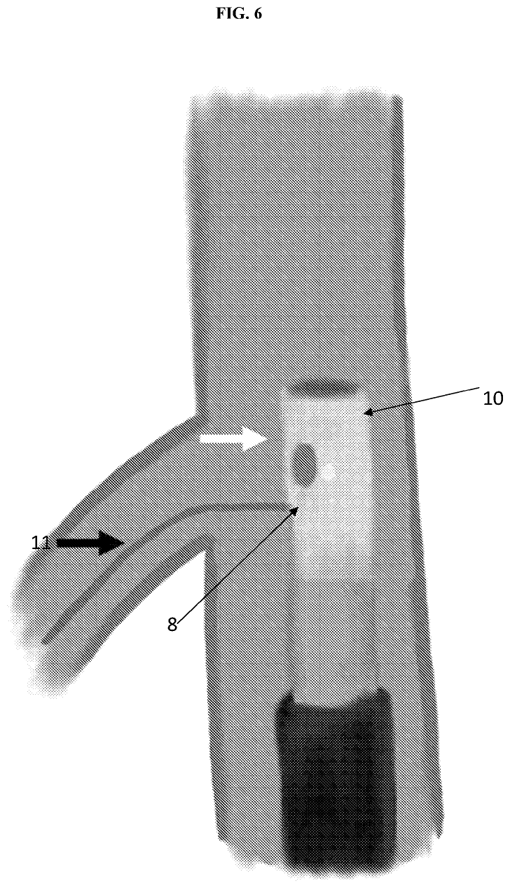

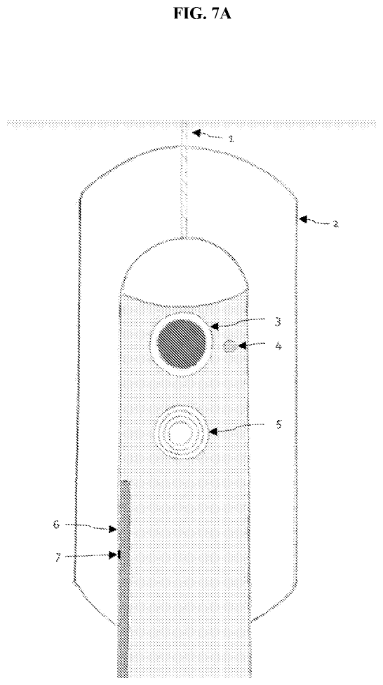

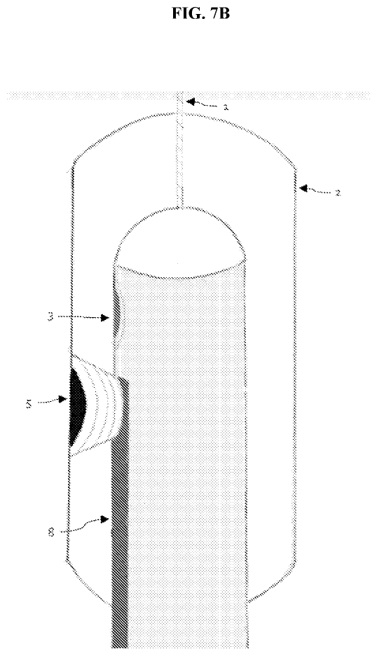

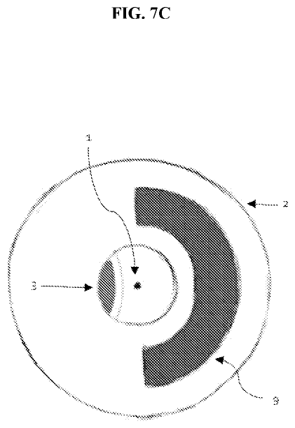

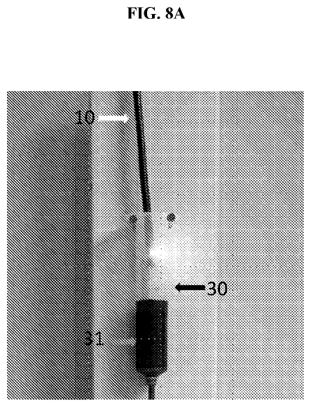

FIGS. 1-7 herein depict an exemplary embodiment of the present technology. The figures variously depict: deployment of a guidewire (1) through a deliver sheath (20) (FIG. 1); advancement of an endoscope (10) comprising a balloon (2), camera (3), light source (4), and working channel opening (5) from a delivery sheath (20) along a guidewire (1) (FIG. 2); extension of a working channel (8) from an endoscope (10) comprising a balloon (2), camera (3), light source (4) (FIG. 3); the distal end of an endoscope (10) comprising a camera (3), light source (4), and working channel (8) (FIG. 4); extension of a working channel wire (11) from a working channel (8) on an endoscope (10) comprising a filled balloon (FIG. 5); placement of a working channel wire (11) from a working channel opening (8) on an endoscope (10) (FIG. 6); a primary side view of an exemplary endoscope depicting a guidewire (1), balloon (2), camera (3), light source (4), working channel opening (5), channel to inflate/deflate the balloon (6), and opening to inflate/deflate the balloon (7) (FIG. 7A); an orthogonal side view of an exemplary endoscope depicting a guidewire (1), balloon (2), camera (3), and working channel (8), (FIG. 7B); and a view of the distal tip of an exemplary endoscope depicting a guidewire (1), balloon (2), camera (3), and channel through the balloon to allow blood flow (9), (FIG. 7C).

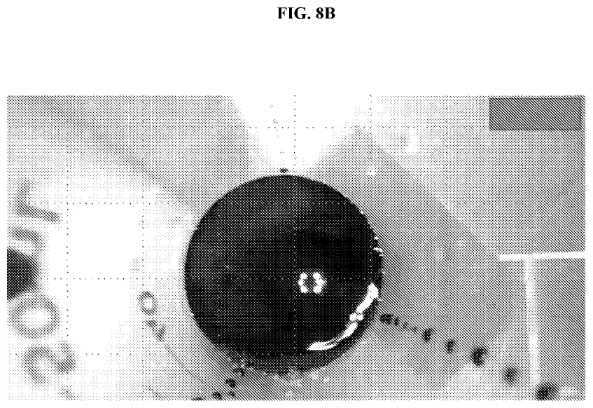

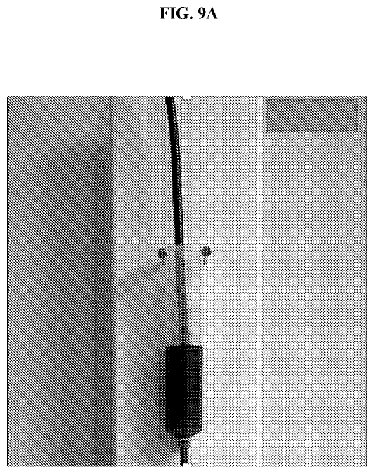

FIG. 8A depicts an image of an exemplary endoscope (10) within a syringe (30) (simulating a blood vessel) that is partially filled with an opaque liquid (31) (simulating blood). FIG. 8B depicts an image of the light from the endoscope through the opaque liquid.

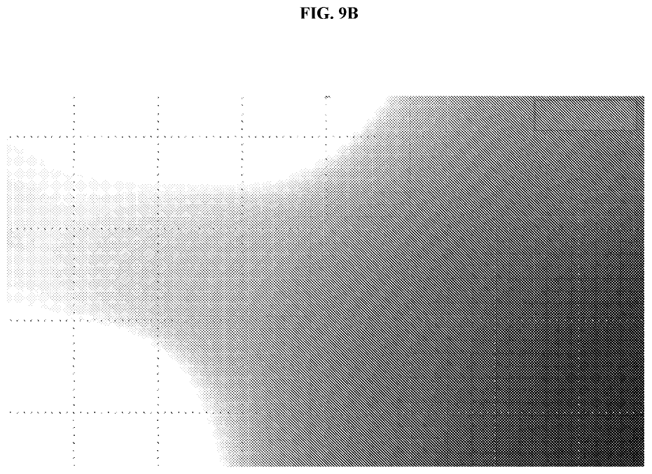

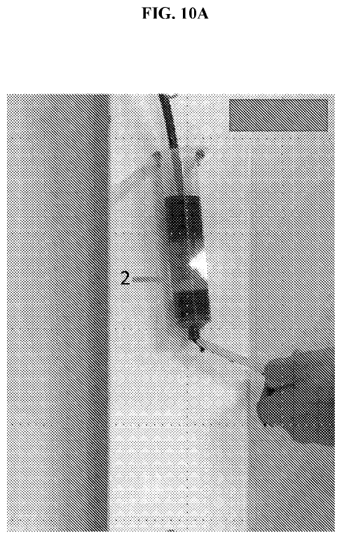

FIG. 9A depicts an image of an exemplary endoscope with the distal end advanced into the opaque liquid. FIG. 9B depicts an image taken from the camera of the endoscope within the opaque liquid without the balloon deployed.

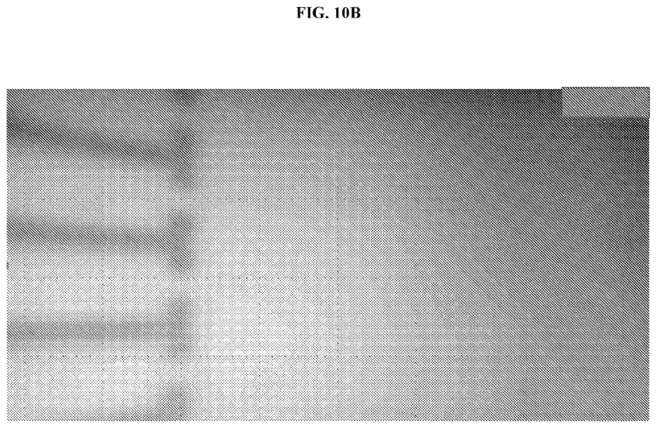

FIG. 10A depicts an image of an exemplary endoscope with the distal end within the opaque liquid and the balloon (2) filled with a transparent fluid. FIG. 10B depicts an image taken from the camera of the endoscope within the opaque liquid with the balloon deployed.

1. An endoscope comprising a main body with a primary axis and a distal end; wherein the distal end is configured for insertion into a subject and placement adjacent to a treatment or diagnostic site within the subject; wherein the distal end comprises a camera and a light source; wherein the distal end comprises a transparent balloon; wherein the endoscope further comprises a channel through the main body capable of delivering a transparent fluid to fill the balloon; wherein when the balloon is filled with the transparent fluid, the balloon is configured to occupy a space between the camera the treatment or diagnostic site and an operator of the endoscope is capable of visualizing the treatment or diagnostic site through the balloon with the camera.

2. The endoscope of claim 1, wherein the distal end of the endoscope comprises the terminal 1-4 cm of the endoscope.

3. The endoscope of claim 1, wherein the camera is oriented orthogonally to the primary axis of the endoscope.

4. The endoscope of claim 1, wherein the balloon is oriented orthogonally to the primary axis of the endoscope.

5. The endoscope of claim 4, wherein the balloon is on the same side of the endoscope as the camera.

6. The endoscope of claim 1, wherein the balloon surrounds the periphery of the distal end of the endoscope.

7. The endoscope of claim 1, wherein the light source is oriented orthogonally to the primary axis of the endoscope.

8. The endoscope of claim 7, wherein the light is oriented in the same direction as the camera.

9. The endoscope of claim 1, wherein the distal end further comprises one or more ports oriented orthogonally to the primary axis.

10. The endoscope of claim 9, further comprising a working channel extending from a port and through the balloon.

11. The endoscope of claim 10, wherein wires, instruments, devices, therapeutics, and/or materials can be transported from the interior of the endoscope to the treatment or diagnostic site through the working channel.

12. The endoscope of claim 10, wherein biological samples can be transported from the treatment or diagnostic site to the interior of the main body of the endoscope through the working channel.

13. The endoscope of claim 10, wherein the working channel is oriented orthogonally to the primary axis.

14. The endoscope of claim 10, wherein the working channel is conical or funnel shaped, having a distal end oriented away from the main body with a greater diameter than a proximal end oriented toward the main body.

15. The endoscope of claim 14, wherein the working channel comprises one or more non-compliant rings oriented along its length to prevent collapse of the working channel under pressure from the filled balloon.

16. The endoscope of claim 1, wherein the camera is positioned at the distal tip of the endoscope and oriented to view beyond the distal end of the endoscope.

17. The endoscope of claim 1, wherein the balloon is positioned at the distal tip of the endoscope and oriented to be filled beyond the distal end of the endoscope.

18. The endoscope of claim 1, wherein the light source is positioned at the distal tip of the endoscope and oriented to illuminate the treatment site beyond the distal end of the endoscope.

19. The endoscope of claim 18, wherein the light is oriented in the same direction as the camera.

20. The endoscope of claim 16, wherein the distal end further comprises one or more ports positioned at the distal tip of the endoscope.

21. The endoscope of claim 20, further comprising a working channel extending from a port and through the balloon.

22. The endoscope of claim 21, wherein wires, instruments, devices, therapeutics, and/or materials can be transported from the interior of the endoscope to the treatment or diagnostic site through the working channel.

23. The endoscope of claim 21, wherein biological samples can be transported from the treatment or diagnostic site to the interior of the main body of the endoscope through the working channel.

24. The endoscope of claim 21, wherein the working channel is oriented substantially parallel to the primary axis.

25. The endoscope of claim 21, wherein the working channel is conical or funnel shaped, having a distal end oriented away from the main body with a greater diameter than a proximal end oriented toward the main body.

26. The endoscope of claim 25, wherein the working channel comprises one or more non-compliant rings oriented along its length to prevent collapse of the working channel under pressure from the filled balloon.

27. A method of viewing a treatment or diagnostic site within a blood vessel or cardiac location of a subject, the method comprising:
(a) placing the distal end of an endoscope of one of claims 1-26 adjacent to the treatment or diagnostic site;
(b) filling the balloon with clear fluid, wherein the balloon fills the space between the endoscope and the treatment or diagnostic site;
(c) illuminating the treatment or diagnostic site with the light source; and
(d) observing the treatment or diagnostic site through the filled balloon with the camera.

28. The endoscope of claim 27, wherein the blood vessel is a vein.

29. The endoscope of claim 27, wherein the blood vessel is an artery.

30. The endoscope of claim 27, wherein when the balloon is filled, the balloon fills the space around the periphery of the endoscope.
